(19) United States
(12) Patent Application Publication
(10) Pub. No.: US 2022/0002923 A1
(43) Pub. Date: Jan. 6, 2022

(54) COMPOSITION FOR MAKING SPUN BOND NONWOVEN FABRIC

(71) Applicant: SABIC GLOBAL TECHNOLOGIES B.V., BERGEN OP ZOOM (NL)

(72) Inventors: Abderrahman MEDDAD, Geleen (NL); Mansour AL-OTAIBI, Geleen (NL); Majed Nabil SAADAWI, Geleen (NL); Jalal BAHRI, Geleen (NL)

(21) Appl. No.: 17/288,802

(22) PCT Filed: Nov. 15, 2019
(86) PCT No.: PCT/EP2019/081451

§ 371 Date: Apr. 26, 2021

(30) Foreign Application Priority Data
Nov. 23, 2018 (EP) 18208008.5

(51) Int. Cl. D04H 3/007; C08L 23/10; D01F 6/46; D04H 3/16

(52) U.S. Cl. CPC D04H 3/007; C08L 23/10; D01F 6/46; D04H 3/16; D10B 2501/04; D10B 2321/022; D10B 2321/021; D10B 2509/02; C08L 2203/12

(57) ABSTRACT

The invention relates to a composition comprising (A) a propylene-based polymer, (B) a C10-C30 aliphatic carboxylic acid amide and (C) a homopolymer or a copolymer of butene-1, wherein the amount of (B) is 1000 to 5000 ppm based on the total composition and the amount of (C) is 5000 to 50000 ppm based on the total composition.

The present invention relates to a polypropylene composition suitable for making fibers.

The invention further relates to fibers made of such polypropylene composition and spun bond nonwoven fabrics made of such fibers.

Spun bond non-woven fabric has been widely used in many applications due to its excellent mechanical properties such as high tensile strength and air permeability. It also ensures the efficient production of fabric based on continuously spun fibers.

Polyamide and polyester have been used for making such spun bond non-woven fabrics. Polypropylene is becoming increasingly prominent within the family of polymeric materials used for spun bond non-woven fabrics.

There have been attempts to impart softness to polypropylene fibers. For example, U.S. Pat. No. 6,740,609 discloses use of a blend of stearamide and erucamide in a polypropylene in an amount of at least 0.02% as a melt additive. US2003157859 discloses a polyolefin resin-based non-woven fabric containing erucamide in an amount of 0.05 to 1.0 wt %, wherein its static friction coefficient is 0.1 to 0.4. U.S. Pat. No. 5,244,724 discloses a fibrous nonwoven web made from a blend of polypropylene with polybutene and/or LLDPE and glycerol monostearate added as an antistatic agent.

There is still a demand in the art for a composition which can be used to produce fibers for making a spun bond nonwoven fabric which has a high softness. Preferably, the composition can be made into fibers with a high throughput.

Accordingly, the present invention provides a composition comprising (A) a propylene-based polymer, (B) a C10-C30 aliphatic carboxylic acid amide and (C) a homopolymer or a copolymer of 1-butene, wherein the amount of (B) is 1000 to 5000 ppm based on the total composition and the amount of (C) is 5000 to 50000 ppm based on the total composition.

It was surprisingly found that spun bond nonwoven fabrics made from fibers made of the composition according to the invention has a high softness. The fabric further exhibits an acceptable tensile strength and elongation at break.

(A) propylene-Based Polymer

Preferably, the amount of the component (A) is at least 90.0 wt %, preferably at least 95.0 wt %, for example 96.0 to 99.0 wt %, with respect to the total composition.

The component (A) may be a propylene homopolymer or a propylene a-olefin random copolymer. The random copolymer consists of at least 70.0 wt % of propylene-derived units and up to 30.0 wt % of comonomer-derived units, based on the total weight of the random copolymer. For example, the amount of the comonomer-derived units based on the total weight of the random copolymer is 1.0 wt % to 20.0 wt %, 2.0 wt % to 10.0 wt % or 3.0 to 5.0 wt %.

Preferably, the comonomer is selected from the group consisting of ethylene, 1-butene, 1-pentene, 4-methyl-1-pentene, 1-hexene, 1-heptene and 1-octene. Most preferably, the comonomer is ethylene.

Preferably, the component (A) has a melt flow rate as measured according to ASTM D1238-13 (2.16 kg/230° C.) of 5.0 to 100 dg/min, for example 10.0 to 50.0 dg/min or 15.0 to 40.0 dg/min.

(B) Amide

The composition according to the invention also comprises a C10-C30 aliphatic carboxylic acid amide. A C10-C30 aliphatic carboxylic acid amide has an amide group CONH₂ group and a long alkyl tail. The C10-C30 carboxylic acid amide is represented by the formula R¹—CONH₂, wherein R¹ is a linear or branched C9-C29 alkyl group. The C10-C30 aliphatic carboxylic acid amide can be saturated C10-30 carboxylic acid amides or unsaturated C10-C30 carboxylic acid amides or mixtures thereof. In the unsaturated carboxylic acid amides at least one carbon-carbon double bond is present in the long alkyl tail.

Examples of saturated carboxylic acid amides are stearamide, palmitamide, cocamide, lauricamide, myristamide, capricamide, tallowamide, myristicamide, margaric (daturic) amide, arachidic amide, behenic amide, lignoceric amide, cerotic amide, montanic amide, melissic amide, lacceroic amide, ceromelissic (psyllic) amide, geddic amide and 9-octadecen amide.

Examples of unsaturated carboxylic acid amides are oleamide, linoleic amide, erucamide, myristoleic amide, palmitoleicamide, sapienic amide, elaidic amide, vaccenic amide, arachidonic amide, eicosapentaenoic amide and decosahexaenoic amide.

The number of carbon atoms in the carboxylic acid amides is 10-30, preferably 12-28, more preferably 14-26, most preferably 16-24.

The carboxylic acid amides are preferably unsaturated C10-C30 carboxylic acid amides, more preferably the carboxylic acid amides are chosen from erucamide and oleamide.

The amount of amide (B) in the composition is preferably between 1000 to 5000 ppm, more preferably 1200 to 3000 ppm, more preferably 1500 to 2500 ppm, with respect to the total composition.

(C) polymer of butene-1

The composition according to the invention also comprises a homopolymer or a copolymer of butene-1. The amount of the comonomer-derived units based on the total weight of the butene-1 copolymer is e.g. 0 to 20.0 wt % or 1.0 to 15.0 wt %.

The comonomer in the butene-1 copolymer is preferably selected from ethylene, propylene, 4-methyl-1-pentene and octene-1. Preferably, the comonomer in the butene-1 copolymer is ethylene or propylene, most preferably ethylene.

Preferably, (C) has a melt flow rate determined by ISO1133-1(2011) of 0.1 to 10 dg/min, preferably 1.0 to 5.0 dg/min.

A preferred example of (C) is a homopolymer of butene-1 having a melt flow rate determined by ISO1133-1(2011) (2.16 kg/190° C./) of 0.1 to 10 dg/min. A commercially available example of such homopolymer of butene-1 is Toppyl PB 0110M available from Lyondellbasell.

A preferred example of (C) is a copolymer of butene-1 with ethylene having a melt flow rate determined by ISO1133-1(2011) (2.16 kg/190° C./) of 0.1 to 10 dg/min. A commercially available example of such copolymer of butene-1 is PB 8220M available from Lyondellbasell.

The amount of (C) in the composition is 5000 to 50000 ppm, preferably 8000 to 40000 ppm, more preferably 10000 to 30000 ppm, with respect to the total composition.

(D) additives

The composition according to the invention may further comprise (D) additives. The additives may include nucleating agents, stabilisers, e.g. heat stabilisers, anti-oxidants, UV stabilizers; colorants, like pigments and dyes; clarifiers; surface tension modifiers; lubricants; mould-release agents; flow improving agents; plasticizers and anti-static agents.

The amount of the component (D) may be 0 to 10 wt %, for example 0.03 to 5.0 wt %, 0.05 to 1.0 wt % or 0.10 to 0.50 wt %, with respect to the total composition.

Composition

The sum of all components added in the process of the invention to form the composition comprising (A), (B), (C) and the optional component (D) should add up to 100% by weight of the total composition.

Preferably, the total of components (A), (B) and (C) is at least 90.0 wt %, at least 95.0 wt %, at least 98.0 wt % or at least 99.0 wt % of the total composition.

Process for Making Composition

The composition of the invention may be obtained by a process comprising melt-mixing (A), (B), (C) and optionally (D) by using any suitable means. Accordingly, the invention further relates to a process for the preparation of the composition according to the invention comprising melt mixing (A), (B), (C) and optionally (D). Preferably, the composition of the invention is made in a form that allows easy processing into a shaped article in a subsequent step, like in pellet or granular form. Preferably, the composition of the invention is in pellet or granular form as obtained by mixing all components in an apparatus like an extruder; the advantage being a composition with homogeneous and well-defined concentrations of the additives.

With melt-mixing is meant that the components (B) and (C) and optionally (D) are mixed with (A) at a temperature that exceeds the melting point of (A). Melt-mixing may be done using techniques known to the skilled person, for example in an extruder. Generally, in the process of the invention, melt-mixing is performed at a temperature in the range from 170-300° C.

Suitable conditions for melt-mixing, such as temperature, pressure, amount of shear, screw speed and screw design when an extruder is used are known to the skilled person.

When using an extruder, a conventional extruder such as a twin-screw extruder may be used. The temperature can vary through the different zones of the extruder as required. For example, the temperature may vary from 100° C. in the feed zone to 300° C. at the die. Preferably, the temperature in the extruder varies from 200 to 265° C. Likewise, the screw speed of the extruder may be varied as needed. Typical screw speed is in the range from about 100 rpm to about 400 rpm.

Properties of Composition

The composition may have a melt flow rate as measured according to ASTM D1238-13 (2.16 kg/230° C.) of 5.0 to 100 dg/min, for example 10.0 to 50.0 dg/min or 15.0 to 40.0 dg/min.

Preferably, the composition has a flexural modulus as measured according to ASTM D790 A of at most 2000 MPa, preferably at most 1500 MPa.

Preferably, the composition has a Rockwell hardness (L) as measured according to ASTM D785 of at most 50, preferably at most 40.

Preferably, the composition has an Izod impact strength as measured according to ASTM D256-10e1 at 23° C. of at least 20 J/m².

The invention further relates to fibers made of the composition according to the invention.

In some embodiments, the fibers have an average diameter of about 5 to 20 μm.

In some embodiments, the fibers are formed into a yarn having a density of 1000 to 2500 denier.

The invention further relates to a spun bond nonwoven fabric made using the fibers according to the invention.

The invention further relates to an article comprising the spun bond nonwoven fabric according to the invention. Suitable examples of the article include liners for sanitary articles, such as disposable diapers and feminine hygiene products and in protective apparel.

It is noted that the invention relates to all possible combinations of features described herein, preferred in particular are those combinations of features that are present in the claims. It will therefore be appreciated that all combinations of features relating to the composition according to the invention; all combinations of features relating to the process according to the invention and all combinations of features relating to the composition according to the invention and features relating to the process according to the invention are described herein.

It is further noted that the term ‘comprising’ does not exclude the presence of other elements. However, it is also to be understood that a description on a product/composition comprising certain components also discloses a product/composition consisting of these components. The product/composition consisting of these components may be advantageous in that it offers a simpler, more economical process for the preparation of the product/composition. Similarly, it is also to be understood that a description on a process comprising certain steps also discloses a process consisting of these steps. The process consisting of these steps may be advantageous in that it offers a simpler, more economical process.

When values are mentioned for a lower limit and an upper limit for a parameter, ranges made by the combinations of the values of the lower limit and the values of the upper limit are also understood to be disclosed.

The invention is now elucidated by way of the following examples, without however being limited thereto.

### EXAMPLES

Molded Composition

A propylene homopolymer having an MFR of 25 dg/min according to ASTM D1238-13 (2.16 kg/230° C.) (PP511A available from Sabic) was blended with components as shown in Tables 1 and 2. The amounts are wt ppm with respect to the total composition. In addition to the additives shown in Table 2, Ex 1-11 contain Irganox 3114 (400 ppm), Irgafos 168 (800 ppm) and Calcium Stearate (350 ppm).

Each of the samples was melt compounded on a twin-screw compounder KraussMaffei (KM) with 25 mm in diameter at melt temperature 226° C. and screw speed of 100 rpm. S1 to S7 were blended by a Henshel mixer for 15 minutes before extrusion. S8-S11 were blended by a V-blender.

Samples for measuring the flexural modulus, Izod impact strength and Rockwell hardness were subsequently injection molded by using Battenfield injection molding machine with a general-purpose screw diameter.

Flexural modulus was measured according to ASTM D790 A.

Rockwell hardness (L) was measured according to ASTM D785.

Izod impact strength was measured according to ASTM D256-10e1.

Melt flow rate as measured according to ASTM D1238-13 (2.16 kg/230° C.).

TABLE 1

| | | |
|---|---|---|
| Additive 1 | Talc | Jetfine ® 3 C A from Imerys Tacl |
| Additive 2 | IPC | Heterophasic propylene copolymer with a matrix of |
| | | propylene homopolymer and a dispersed phase of |
| | | propylene-ethylene copolymer, MFR (ASTM D1230, |
| | | 230° C., 2.16 kg) of 16 dg/min (SABIC 48MK40) |
| Additive 3 | NA-27 | Nucleating agent available from ADK as STAB NA-27 |
| Additive 4 | HDPE | HDPE having MFR (ASTM D1230, 190° C., 2.16 kg) of 20 |
| | | dg/min (SABIC M200056) |
| Additive 5 | HPN20E | Nucleating agent available from Milliken as Hyperform ® |
| | | HPN-20E |
| Additive 6 | Erucamide | Crodamide ER from CRODA |
| Additive 7 | PB-1 | Toppyl PB 8220M from LyonDell Basell |
| Additive 8 | L-MODU | Low molecular weight and low modulus polypropylene |
| | S901 | available from Idemitsu Kosan as L-MODU S901 |

TABLE 2

| | 1 | 2 | 3 | 4 | 5 | 6 | 7 | 8 | 9 | 10 | 11 |
|---|---|---|---|---|---|---|---|---|---|---|---|
| Additive 1 | | 500 | | | | | | | | | |
| Additive 2 | | | 1000 | | 1000 | | | | | | |
| Additive 3 | | | | 500 | 500 | | | | | | |
| Additive 4 | | | | | | 500 | 500 | | | | |
| Additive 5 | | | | | | | 250 | | | | |
| Additive 6 | | | 1000 | | 1000 | | | 2000 | 2000 | 2000 | 2000 |
| Additive 7 | | | | | | | | 1500 | 10000 | 25000 | |
| Additive 8 | | | | | | | | | | | 100000 |
| Flexural | 2083 | 2157 | 2028 | 2276 | 2358 | 2124 | 2410 | 2158 | 1996 | 1236 | 1112 |
| modulus | | | | | | | | | | | |
| (MPa) | | | | | | | | | | | |
| Rockwell | 50.67 | 52.75 | 52 | 51.83 | 53.92 | 51.58 | 56.83 | 53.42 | 50.75 | 48.08 | 41 |
| Hardness | | | | | | | | | | | |
| (L) (HRL) | | | | | | | | | | | |

Sample S-11 is a grade having a high softness, containing a relatively large amount of low molecular weight polypropylene.

It can be understood that the addition of Erucamide and 25000 ppm of PB-1 (S-10) leads to a high softness, which can be seen by the low flexural modulus and the low Rockwell hardness.

Fibers

Bulked-continuous filaments were manufactured using a lab scale BCF line “Reiter”. The processing parameters were set to produce a yarn titer counts 1200 denier/80 filaments.

The melting temperature was set at 235° C., and the take up speed was at about 2000 m/min.

Tenacity and elongation at break were measured according to ASTM D2256.

TABLE 3

| | Tenacity (cN/tex) | Elongation at break (%) |
|---|---|---|
| S-1 | 14.6 | 26 |
| S-10 | 15.3 | 28 |
| S-11 | 15.4 | 25 |
| S-15 | 15.2 | 27 |

Sample without any of additive 1-8 (S-1) has a slightly lower tenacity and similar elongation at break compared to those of S-9, S-10 and S-11.

Spunbond Nonwoven Fabrics

Spunbond nonwoven fabrics were made from S-1 and S-10 on a 1.1 m wide Reicofil 4 line with two beams. The trial was run at a throughput of about 1200 kg/hour/meter using the processing parameters given in the Table 4. The nonwoven was thermally bonded using a new embossed roll pattern.

TABLE 4

| | |
|---|---|
| Fabric weight (g/m2) | 13 |
| Line speed (m/min) | 800 |
| Throughput (kg/h) | 2200 |
| Pressure Cabin (Pa) | 4500 |
| Suction air fan (rpm) | 2400 |
| Process air temperature (° C.) | 20 |
| Die pressure (bar) | 23 |
| Melt temperature (° C.) | 248 |

The stiffness, tensile strength and elongation at break were measured.

The stiffness was measured using a commercial testing equipment known as “Handle-O-Meter” test as specified in operating manual on Handle-O-Meter model number 211-5 from the Thwing-Albert Instrument Co.

The tensile strength was measured according to Edana standard WSP 110.4.

The elongation at break was measured according to Edana standard WSP 110.4.

Results are summarized in Table 5.

TABLE 5

| | | | Tensile | Tensile | Elongation | Elongation |
|---|---|---|---|---|---|---|
| | Stiffness | Stiffness | strength | strength | at break | at break |
| | (MD) (mN) | (CD) (mN) | (MD) (N/cm) | (CD) (N/cm) | (MD) (%) | (CD) (%) |
| S-1 | 36.6 | 19.2 | 5.66 | 3 | 59.4 | 75.01 |
| S-10 | 31 | 15.5 | 5.88 | 2.82 | 80.9 | 94.85 |

MD denotes machine direction and CD denotes cross direction.

The fabric made from the composition of S-10 exhibits a lower stiffness than that made from S-1, which indicates that the fabric of S-10 is softer than the fabric of S-1.

The fabric made from the composition of S-10 exhibits a similar tensile strength and a higher elongation at break compared to the fabric of S-1.

1. A composition comprising
(A) a propylene-based polymer, (B) a C10-C30 aliphatic carboxylic acid amide and (C) a homopolymer or a copolymer of butene-1, wherein the amount of (B) is 1000 to 5000 ppm based on the total composition and the amount of (C) is 5000 to 50000 ppm based on the total composition.

2. The composition according to claim 1, wherein (B) is an unsaturated C10-C30 aliphatic carboxylic acid amide.

3. The composition according to claim 1, wherein the amount of (B) is 1200 to 3000 ppm.

4. The composition according to claim 1, wherein (C) is a copolymer of butene-1 with a comonomer selected from ethylene, propylene, 4-methyl-1-pentene and octene-1.

5. The composition according to claim 1, wherein (C) has a melt flow rate determined by ISO1133-1:2011 (2.16 kg/190° C.) of 0.1 to 10 dg/min.

6. The composition according to claim 1, wherein the amount of (C) is 8000 to 40000 ppm, with respect to the total composition.

7. The composition according to claim 1, wherein (A) is a propylene homopolymer or a propylene random copolymer consisting of at least 70.0 wt % of propylene-derived units and up to 30.0 wt % of comonomer-derived units based on the total weight of the random copolymer, wherein the comonomer is selected from the group consisting of ethylene and α-olefins having 4-10 carbon atoms.

8. The composition according to claim 1, wherein (A) has a melt flow rate as measured according to ASTM D1238-13 (2.16 kg/230° C.) of 5.0 to 100 dg/min.

9. The composition according to claim 1, wherein the total of (A), (B) and (C) is at least 90.0 wt % of the total composition.

10. The composition according to claim 1, wherein the composition has a melt flow rate as measured according to ASTM D1238-13 (2.16 kg/230° C.) of 5.0 to 100 dg/min.

11. The composition according to claim 1, wherein the composition has a flexural modulus as measured according to ASTM D790 A of at most 2000MPa.

12. The composition according to claim 1, wherein the composition has a Rockwell hardness (L) as measured according to ASTM D785-08 of at most 50.

13. Fibers made of the composition according to claim 1.

14. A spun bond nonwoven fabric made using the fibers according to claim 13.

15. An article comprising the spun bond nonwoven fabric according to claim 14.

16. The article according to claim 15, wherein the article is selected from liners for sanitary articles and liners in protective apparel.

17. The composition according to claim 1, wherein (B) is selected from erucamide and oleamide.

18. The composition according to claim 1, wherein the amount of (B) is 1500 to 2500 ppm, with respect to the total composition.

19. The composition according to claim 1, wherein (C) is a copolymer of butene-1 with ethylene.

20. The composition according to claim 1, wherein the amount of (C) is 10000 to 30000 ppm, with respect to the total composition.
